(19) United States
(12) Patent Application Publication
(10) Pub. No.: US 2005/0064044 A1
(43) Pub. Date: Mar. 24, 2005

(54) GSK-3beta inhibitors in the treatment of bone-related diseases

(76) Inventors: Georges Rawadi, Paris (FR); Sergio Roman, Paris (FR); Philippe Clement-Lacroix, Le Pre Saint Gervais (FR)

Correspondence Address: HELLER EHRMAN WHITE & MCAULIFFE LLP, 1666 K STREET,NW, SUITE 300, WASHINGTON, DC, 20006, US

(21) Appl. No.: 10/665,816

(22) Filed: Sep. 19, 2003

(51) Int. Cl. A61K 33/00; A61K 33/24; A61K 31/503; A61K 31/498

(52) U.S. Cl. 424/617; 514/248; 514/250; 514/414; 514/425; 424/722

(57) ABSTRACT

The present invention relates to methods for preventing and/or treating bone-related diseases in mammals, especially humans, using GSK-3β inhibitors. The invention also concerns methods for selecting in vitro and/or in vivo compounds useful for preventing and/or treating bone-related diseases in mammals, including humans.

### TECHNICAL FIELD OF THE INVENTION

The present invention relates to prophylactic and/or therapeutic treatments of bone-related diseases in mammals.

In this respect, the invention concerns new medical applications of GSK-3β inhibitors.

The present invention is thus related to methods for preventing and/or treating bone-related diseases in mammals, especially humans, using GSK-3β inhibitors.

The invention is also directed to methods for selecting in vitro and/or in vivo compounds useful for preventing and/or treating bone-related diseases in mammals, including humans.

### BACKGROUND AND PRIOR ART

Glycogen synthase kinase 3 (GSK-3) is a multifunctional serine/threonine kinase (see the commentary of Doble and Woodgett, 2003).

There are two mammalian GSK-3 isoforms encoded by distinct genes: GSK-3α and GSK-3β (Woodgett, 1990). GSK-3α has a mass of 51 kDa, whereas GSK-3β is a protein of 47 kDa. The difference in size is due to a glycine-rich extension at the N-terminus of GSK-3α. Although highly homologous within their kinase domains (98% identity), the two gene products share only 36% identity in the last 76 C-terminal residues. Moreover, GSK-3α and GSK-3β, although structurally related, are not functionally identical (Doble and Woodgett, 2003).

Homologues of GSK-3 exist in all eukaryotes examined to date and display a high degree of homology (for a review, see Ali et al., 2001).

Beyond its first evidenced role in glycogen metabolism, GSK-3 acts as a downstream regulatory switch that determines the output of numerous signalling pathways initiated by diverse stimuli (reviewed in Frame and Cohen, 2001). The pathways in which GSK-3 acts as a key regulator, when dysregulated, have been implicated in the development of human diseases such as diabetes, Alzheimer's disease, bipolar disorder and cancer.

Given its involvement in many pathophysiological processes and diseases, GSK-3 appears to be an interesting candidate target for drug development.

However, its involvement in multiple pathways also raises the issue of selectivity. For example, although inhibition of GSK-3 may be desirable for a given therapeutic purpose, it could have deleterious implications for another, e.g., it is assumed to accelerate hyperplasia by deregulating β-catenin.

The Wnts are a family of secreted, cysteine-rich, glycosylated, protein ligands that influence cell growth, differentiation, migration and fate (reviewed in Miller, 2002).

One of the pathways regulated by Wnt molecules is the Wnt/β catenin pathway (Huelsken and Behrens, 2002). In unstimulated cells, GSK-3β phosphorylates the N-terminal domain of β-catenin, thereby targeting it for ubiquitylation and proteasomal degradation. Exposure of cells to Wnts leads to inactivation of GSK-3β and results in the dephosphorylation of β-catenin, which thus escapes the ubiquitylation-dependent destruction machinery (van Noort et al., 2002). Unphosphorylated β-catenin accumulates in the cytoplasm and translocates to the nucleus, where it binds to the effector transcription factors TCF/LEFs, and activates transcription of target genes.

In U.S. Patent Application published under No. 2003/0027151, in the names of Warman et al., loss of function of the Wnt receptor LRP5 is described to lead to osteoporosis. Moreover, a specific mutation in this receptor results in high bone mass.

Osteoporosis is a common medical problem with major morbidity and societal cost. Individuals afflicted with this disease present diminished bone strength as a consequence of low bone mineral content.

Despite the currently available treatments for osteoporosis and, more generally, bone-related diseases, there is still a need for new treatments using drugs that would be efficient, easy to administer, economical to manufacture, and cost-competitive to sell.

In the context of the present invention, given that LRP5 acts as a co-receptor for Wnt proteins, Wnt/β-catenin signalling was tested for its possible involvement in bone formation.

In this respect, the present invention shows that molecules that are capable of stabilizing β-catenin through the inhibition of GSK-3β, some being reviewed for instance in Doble and Woodgett (2003), have a Wnt-like beneficial effect on bone formation, and are thus useful for preventing and/or treating bone-related diseases.

In addition, unlike Wnt proteins, GSK-3β inhibitors do not increase cell proliferation. Consequently, contrary to what was generally assumed, GSK-3β inhibitors advantageously appear not to result in hyperplasia when deregulating β-catenin.

### SUMMARY OF THE INVENTION

It is an object of the present invention to provide methods for preventing and/or treating bone-related diseases in mammals in need of such treatment, using GSK-3β inhibitors.

It is another object of the invention to provide methods for selecting GSK-3β inhibitors useful for preventing and/or treating bone-related diseases in mammals in need of such treatment.

Further objects will be appreciated from a reading of the contents herein.

### BRIEF DESCRIPTION OF THE DRAWINGS

FIG. 1 shows the effect of lithium on a Wnt-signalling luciferase reporter construct in the pluripotent mesenchymal cell line C3H10T1/2.

FIG. 2 shows the effect of lithium on the alkaline phosphatase (ALP) osteoblast differentiation marker in the pluripotent mesenchymal cell line C3H10T1/2.

FIG. 3 shows the bone phenotype of LRP5-knockout mice models at 4 weeks of age. * p<0.05, ** p<0.01, *** p<0.001. BV/TV: bone volume, wt: wild-type, KO: knockout.

FIG. 4 shows the effect of lithium on the bone phenotype of LRP5-knockout mice models at 5 weeks of age. * p<0.05, ** p<0.01, p<0.001. A BV/TV: bone volume. B Tb.N: trabecular number. C Tb.Th: trabecular thickness.

FIG. 5 shows the effect of lithium on the bone phenotype of LRP5-knockout mice models at 4 weeks of age. * p<0.05, ** p<0.01, p<0.001. A BV/TV: bone volume. B Tb.N: trabecular number. C Tb.Th: trabecular thickness.

FIG. 6 shows three-dimensional reconstruction of bone tissue from 4 week-old LRP5-knockout mice treated with vehicle (A and B) or lithium (C and D).

### DETAILED DESCRIPTION OF THE PREFERRED EMBODIMENTS

The term “disease” means herein an alteration of the health of a mammal, due to internal and/or external causes, said alteration becoming apparent through symptoms and resulting in an impairment of one or more biological functions, such as metabolic functions, and/or in one or more lesions in said mammal.

By the term “disorder”, it is meant herein a pathological modification of an organ or of a physical or psychological function in a mammal.

For the purpose of the invention, the terms “alteration”, “impairment”, and “modification” as recited above are synonymous.

Moreover, the terms “disease” and “disorder” are used herein interchangeably, unless otherwise specified.

In the context of the present invention, the expression “bone-related disease” refers to a disorder directly or indirectly affecting bone cells, that gives rise to a condition of clinical relevance for skeletal health.

The mechanisms that give rise to such a disease are diverse and may be mediated by primary pathology affecting bone cells (an example is Paget's disease of bone), or indirectly. Indirect mechanisms include the effects of abnormal endocrine secretion of major calcium and skeletal regulating hormones, including sex hormones (estrogen, androgen, progesterone, and the like). Examples include post-menopausal osteoporosis, primary hyper parathyroidism and Cushing's disease. Bone disease may also arise from the local or systemic effects of cytokines such as in multiple myeloma, periodontal disease.

Intrinsic bone disease may be genetic (e.g., epiphyseal dysplasia) or acquired (e.g., osteomyelitis).

However, in practice, as knowledge of pathophysiology advances, the distinction between intrinsic and metabolic bone diseases becomes increasingly blurred.

Moreover, importantly, pathophysiology of bone disease may also involve target tissues other than bone. An illustrative example is vitamin D deficiency which gives rise to osteomalacia in adults or rickets in childhood.

In this respect, the expression “bone-related diease” encompasses at least disorders of mineral metabolism, disorders of parathyroid hormone (PTH) secretion and/or activity, metabolic bone disorders comprising osteoporosis, vitamin D-related disorders, renal bone diseases, hypophosphatasia, dysplastic disorders, infiltrative disorders, extra-skeletal calcification and ossification, miscellaneous disorders, and the like (for a literature reference, see Baron et al.).

By “disorders of mineral metabolism”, it is meant herein at least hypercalcaemia of diverse causes, hypocalcaemia of diverse causes, hyperphosphataemia, hypophosphataemia, hypermagnesaemia, hyporhagnesaemia, and the like.

Under the expression “disorders of PTH secretion and/or activity” are included for instance hyperparathyroidism, hypoparathyroidism, pseudohypoparathyroidism, and the like.

“Miscellaneous disorders” encompass at least medullary carcinoma, skeletal toxicity syndromes (e.g., aluminium, iron>cadmium, fluorosis), alveolar bone resorption, non-union and fracture repair, bone reconstruction, ischaemic disorders, osteonecrosis, and the like.

A “metabolic bone disorder” includes at least osteoporosis, which may be for instance postmenauposal, involutional, secondary; as well as hypo-remodelling syndromes; and the like.

As used herein, the expression “vitamin D-related disorders” relates at least to nutritional, resistance,-secondary hyperparathyroidism, ectopic 1-alpha-hydroxylase activity, oncogenic, and the like.

By “renal bone disease”, it is meant for instance osteitis fibrosa, osteomalacia, osteosclerosis, osteoporosis, adynamic bone disease, and the like.

“Hypophosphatasia” refers to, for example, hyperphosphatasia, Paget's disease, Engelman's disease, and the like.

“Dysplastic disorders” may be for instance sclerosing bone dysplasias and osteoporosis, fibrous dysplasia, mucopolysaccharidoses, periostoses, ankylosing spondylarthritis, osteochondroses, osteophytosis, Diffuse Osteopathic Skeletal Hyperostosis (DISH), osteogenesis imperfecta, genetic disorders, and the like.

“Infiltrative disorders” include at least primary skeletal neoplasms, secondary skeletal neoplasms, systemic mastocytosis and histiocytosis, sarcoidosis, oxalosis, and the like.

“Extra-skeletal calcification and ossification” may be for example renal bone disease, fibrodysplasia ossificans progressiva, nephrolithiasis, and the like.

In an embodiment of the present invention, a “bone-related disease” is osteoporosis.

According to the invention, the term “mammals” encompasses animals and humans. In an embodiment, a “mammal” is a human.

A “compound” herein refers to any type of molecule, biological or chemical, natural, recombinant or synthetic. For instance, such a compound may be a nucleic acid (e.g., an antisense or sense oligonucleotide including an antisense RNA), a protein, a fatty acid, an antibody, a polysaccharide, a steroid, a purine, a pyrimidine, an organic molecule, a chemical moiety, and the like.

The term “compound” is preferably used herein to refer to a compound which exhibits the function of interest, i.e., the ability to inhibit the GSK-3β biological activity.

In this respect, also encompassed by the term “compound” are fragments, derivatives, structural analogs, and combinations thereof, all of them being functional, i.e., being capable of inhibiting the GSK-3β biological activity.

The above-defined “compound” is also referred to herein as a “GSK-3β inhibitor”.

As used herein, a “molecule” is of any type, biological or chemical, natural, recombinant or synthetic. For instance, such a molecule may be a nucleic acid (e.g., an antisense or sense oligonucleotide including an antisense RNA), a protein, a fatty acid, an antibody, a polysaccharide, a steroid, a purine, a pyrimidine, an organic molecule, a chemical moiety, and the like.

The terms “molecule” and “compound” thus refer to the same structures.

However, as used herein, these terms are not equivalent, since a “compound” is, as defined above, a “GSK-3β inhibitor”, whereas a “molecule” either displays a biological function, which is thus different than the ability to inhibit the GSK-3β biological activity, or it is inert, i.e., it does not have any biological function.

As used herein, the terms “activity” and “active”, and “function” and “functional” are synonymous, respectively. Moreover, the terms and expressions “biological activity”, “biological function”, “activity”, and “function” are also synonymous.

By “inhibiting GSK-3β activity”, it is meant that said GSK-3β activity is “reduced” or “decreased” or “suppressed” or “blocked”. This may reflect, for instance, (i) a decrease in expression or in activity of the GSK-3β-encoding polynucleotide or of the GSK-3β polypeptide; or (ii) a change in the amount of said GSK-3β-encoding polynucleotide or of the GSK-3β polypeptide, in the cellular distribution thereof, in the level of expression thereof, in the type of activity thereof.

As used herein, a “pharmaceutical composition” is equivalent to a “pharmaceutical preparation”, both referring to a “drug” as commonly understood by the skilled artisan in the field of the invention. More precisely, said “pharmaceutical composition” or “pharmaceutical preparation” or “drug” comprises a pharmaceutically acceptable amount of one or more compounds and, optionally, one or more molecules, all of them being generally associated to, or contained in, at least one pharmaceutically acceptable carrier.

The “pharmaceutically effective amount” of an active compound is the amount of said compound that results in amelioration of symptoms in a mammal.

A “pharmaceutically acceptable carrier”, also referred to as an “adjuvant”, is conventional and may easily be chosen by the one skilled in the art, depending on the administration route of the drug under consideration, by relying on the general knowledge in techniques for formulating drugs (see the Remington reference).

According to a first aspect, the present invention relates to a method for preventing and/or treating a bone-related disease in a mammal in need of such treatment, wherein said method comprises:
administering to said mammal an effective amount of a pharmaceutical composition comprising at least one GSK-3β inhibitor.

Such a pharmaceutical composition comprises one or more different GSK-3β inhibitors and, optionally, one or more molecules which, as defined above, do not exhibit the ability to inhibit GSK-3β activity.

For instance, these molecules may only act as adjuvants or carriers, such as polylactic acid, polyglycolic acid, polydioxanone, collagen, albumin, detergent (e.g., polyoxyethylenesorbitan), and the like.

Other useful molecules may have a biological function (hereafter referred to as “biologically-active molecules”), different that the one of GSK-3β inhibitors, but the association of which may be of interest regarding bone formation and protection.

In this respect, biologically-active molecules may be vitamins.

Other useful biologically-active molecules may be molecules that promote tissue growth or infiltration, including bone morphogenic proteins such those described in U.S. Pat. No. 4,761,471 and PCT Publication WO 90/11366, osteogenin (Sampath et al., 1987), and NaF (Tencer et al., 1989).

Yet other biologically-active molecules may be targeting molecules, i.e., molecules that bind to (have affinity with) the tissue of interest. Examples of bone-targeting molecules include tetracyclines; calcein; biphosphonates; polyaspartic acid; polyglutamic acid; aminophosphosugars; peptides known to be associated with the mineral phase of bone such as osteonectin, bone sialoprotein and osteopontin; bone specific antibodies; proteins with bone mineral binding domains; and the like (for example, see Bentz et al. in EP 0512844 and Murakami et al. in EP 0341961).

According to a second aspect, the invention concerns a method for preventing and/or treating a bone-related disease in a mammal in need of such treatment, wherein said method comprises:
administering to said mammal a pharmaceutically effective amount of at least one GSK-3β inhibitor.

For the purpose of determining the pharmaceutically effective amount of GSK-3β inhibitors as defined above, toxicity and therapeutic efficacy of the compounds can be determined by standard pharmaceutical procedures in cell cultures (in vitro) or in experimental animals (in vivo). For example, the LD50 (the dose lethal to 50% of the population), as well as the ED50 (the dose therapeutically effective in 50% of the population) can be determined using methods known in the art.

Accordingly, the data obtained from cell culture assays (in vitro) and/or animal model studies (in vivo) can be used in formulating a range of dosage of these compounds which lies preferably within a range of circulating concentrations that include the ED50 with little or no toxicity.

In the context of the invention, administration of a drug may be performed via any route such as locally, orally, systemically, intravenously, intramuscularly, mucosally, using a patch, using encapsulating or embedding liposomes, microparticles, microcapsules, and the like.

In an embodiment, said at least one GSK-3β inhibitor is selected from lithium, bivalent zinc, beryllium, aloisines, hymenialdisine, indirubins, maleimides, muscarinic agonists, pyrazolo[3,4-b]quinoxalines, 5-aryl-pyrazolo[3,4-b]pyridazines, and functional derivatives thereof (see Doble and Woodgett, 2003; Ortega et al., 2002; Witherington et al., 2003).

In another embodiment, said at least one GSK-3β inhibitor is lithium.

According to a third aspect, the present invention is related to a method for selecting a compound useful for preventing and/or treating a bone-related disease in a mammal in need of such treatment, wherein said method comprises:
a) testing the ability of a candidate compound to inhibit GSK-3β activity in vitro and/or in vivo; and
b) if said candidate compound inhibits GSK-3β activity, selecting said compound.

In an embodiment, this method further comprises purifying the selected compound.

Methods for detecting an inhibition of GSK-3β activity include both in vitro and in vivo procedures (e.g., protein-protein binding assays, biochemical screening assays, immunoassays, cell-based assays, animal model experiments, which are well-characterized in the art). For instance, the person skilled in the art may use only one in vitro and/or one in vivo selection technique. However, in order to strengthen the validity and reproducibility of the results, this person may prefer to use at least two in vitro and/or at least two in vivo selection methods. Examples of in vitro and in vivo procedures for showing an inhibitory activity on GSK-3β are given hereunder. Another example of in vitro model is monitoring the induction of alkaline phosphatase in osteoblast-like cell lines or in primary calvaria cells. Other examples of in vivo models result from inducing osteopenia in rodents after oviariectomy (females) or orchidectomy (males), or in thyro-parathyroidectomized rodents.

The present invention also encompasses the use of at least one GSK-3β inhibitor for the manufacture of a pharmaceutical composition for preventing and/or treating bone-related diseases in a mammal.

In order to fully illustrate the present invention and advantages thereof, the following specific examples are given, it being understood that the same are intended only as illustrative and in no way as limitative.

### EXAMPLES

The following examples illustrate that inhibiting GSK-3β induces osteoblast differentiation markers, thus resulting in increased bone formation.

For this purpose, both cellular in vitro model and animal in vivo model were used.

In vitro model used the pluripotent mesenchymal cell line C3H10T1/2 that is able to differentiate into osteoblasts when triggered with the right compound or protein. The ability of a compound to induce these C3H10T1/2 cells to differentiate into osteoblasts can be monitored by, for instance, measuring the expression of the osteoblast differentiation marker, alkaline phosphatase (ALP).

In vivo model uses LRP5 knock-out animals that show osteopenia phenotype resulting from the absence of the Wnt canonical signalling pathway, said signalling pathway involving GSK-3β. Thanks to this model, the effect of GSK-3β inhibitors on bone mass can be observed.

### Example 1

### Lithium Activates Wnt3a Signalling in C3H10T1/2 Cells

Whether inhibition of GSK-3β in C3H10T1/2 cells leads to Wnt/β-catenin signalling activation was investigated.

C3H10T1/2 cells were transiently transfected using Fugen6 (Boehringer) with a Wnt signalling luciferase reporter construct (van de Wetering et al., 1997). To assess transfection efficacy, 20 ng of pRL-TK (Promega) encoding a Renilla luciferase gene downstream of a minimal HSV-TK promoter was systematically added to the transfection mix. Cells were stimulated with LiCl or with NaCl for 24 h. Cells were lysated and luciferase assays were performed with the Dual Luciferase Assay Kit (Promega) according to the manufacturer's instructions. 10 μl of cell lysate was assayed first for firefly luciferase and then for Renilla luciferase activity. Firefly luciferase activity was normalized to Renilla luciferase activity.

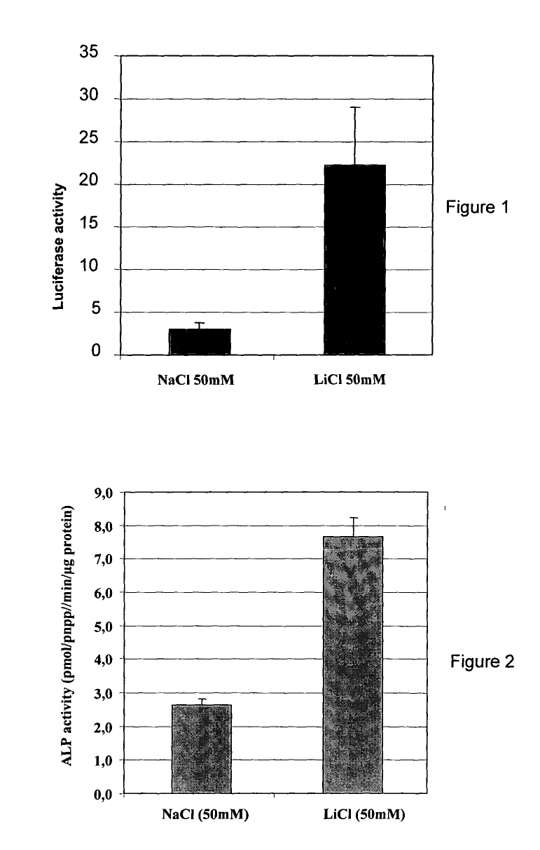

As shown in FIG. 1, lithium was able to activate luciferase expression, thus clearly demonstrating that inhibiting GSK-3β in the pluripotent mensenchymal cell line C3H10T1/2 results in the activation of Wnt3a activity involved in the canonical Wnt signalling.

### Example 2

### Lithium Induces the Expression of Alkaline Phosphatase (ALP) in C3H10T1/2 Cells

Whether inhibition of GSK-3β by LiCl in C3H10T1/2 cells leads to the expression of ALP was investigated.

C3H10T1/2 cells were stimulated with LiCl or with NaCl for 48 h. ALP activity was determined in cell lysates using Alkaline Phosphatase Opt kit (Roche Molecular Biochemicals). Cell lysates were analyzed for protein content using micro-BCA Assay kit (Pierce), and ALP activity was normalized for total protein concentration.

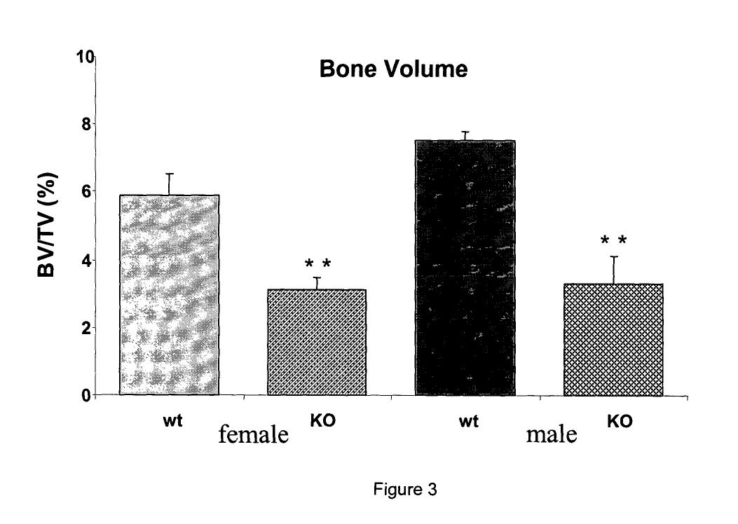

As shown in FIG. 2, lithium is able to stimulate the expression of the ALP osteoblast differentiation marker in the pluripotent mensenchymal cell line C3H10T1/2, thus clearly showing that inhibiting GSK-3β in C3H10T1/2 cells stimulates cells to differentiate into osteoblast lineage.

### Example 3

### Use of LRP5 Knockout Mice as Pharmacological In Vivo Models to Test GSK-3β Inhibitors

3-1—Proof of Concept:

LRP5 knockout mouse model has been described as an osteopenic mouse model (Kato et al., 2002).

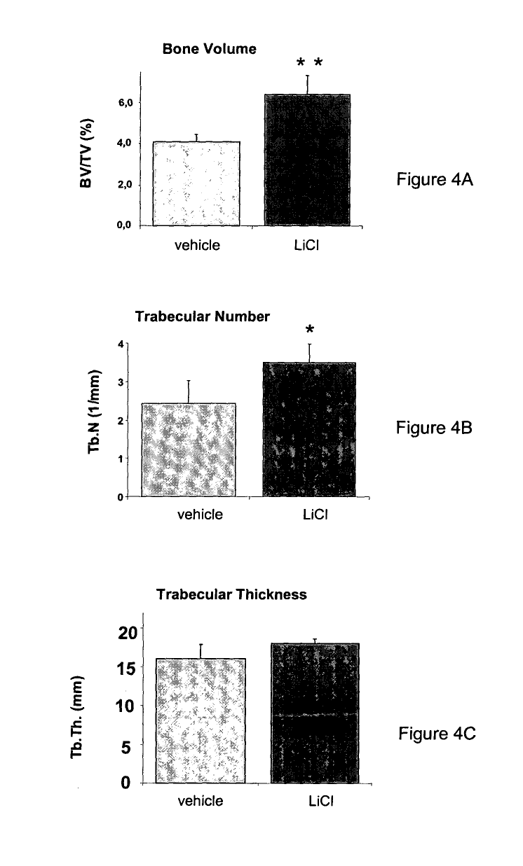

It was observed that, as soon as 4 weeks of age, LRP5 knockout mice present a significant reduction of trabecular bone volume in long bone (FIG. 3).

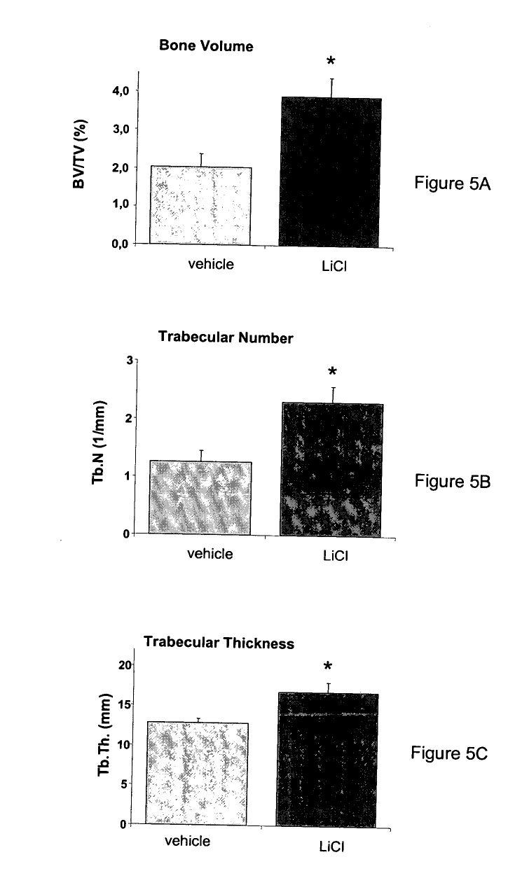

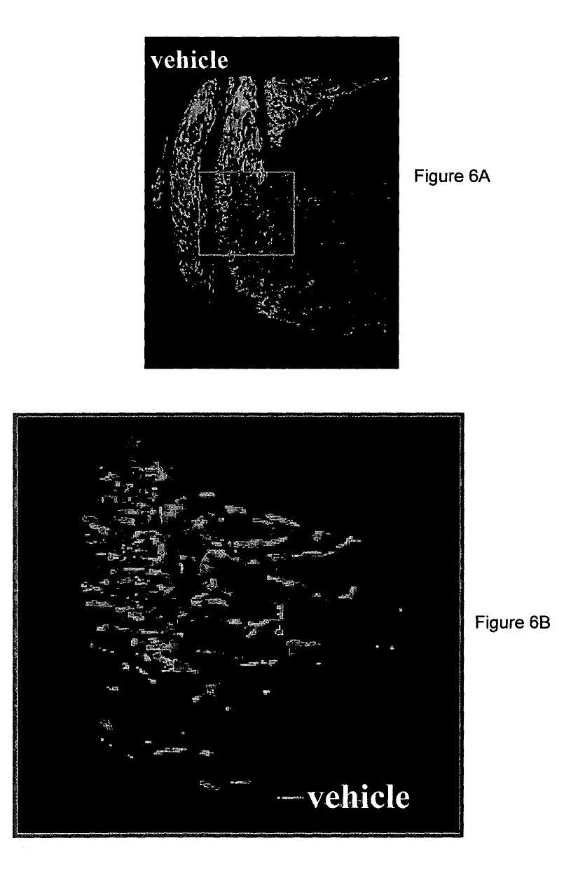

Given that GSK-3β activity was supposed to be under the control of the LRP5 pathway, the bone phenotype was, as shown in FIGS. 4 and 5, partially reversed using a GSK-3β inhibitor such as LiCl.

3-2—Materials and Methods:

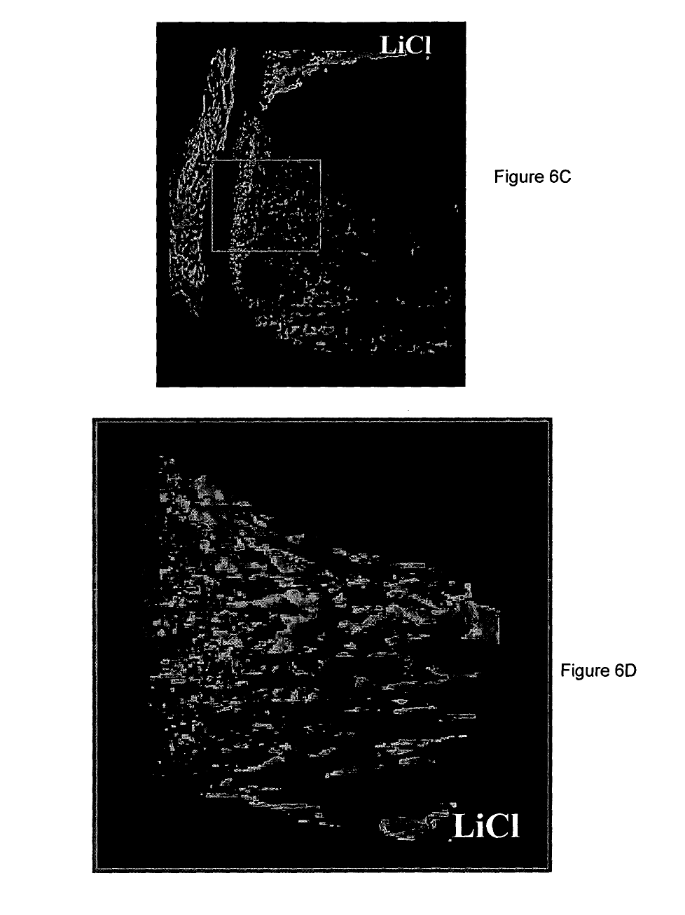

LiCl solution was prepared in distillated water at 55 mg/ml. Compound was administered by micropump Alzet (ref: 1002, Charles Rivers, France) to 2-3 week-old LRP5 knockout (KO) mice for 2 weeks. Tibia were prepared for tomographic analysis (Tomodensitometer Scanco μCT20, Basserdorf, Switszerland). Micro-CT scans of the metaphyseal tibia were performed at an isotropic resolution of 9 μm, to obtain trabecular bone structural parameters. Using a two- and three-dimensional model and a semiautomatic contouring algorithm, three-dimensional bone volume, bone surface, and trabecular thickness were determined (FIG. 6).

Statistical significance was determined by the ANOVA unpaired t'test.

3-3—Bone Phenotype of LRP5-KO Mice at 4 Weeks of Age:

As illustrated in FIG. 3, histological tomodensitometric analysis of metaphyseal trabecular part of tibia from 4 week-old wild-type and LRP5-KO mice shows a similar low bone mass in the mutant mice both gender.

Bone volume was significantly decreased about −47% and −56% in female and male, respectively. Trabecular thickness and trabecular number were also decreased in mutant mice (data not shown).

3-4—Bone Phenotype of LRP5-KO Mice is Restored by LiCL Treatment:

Results of tomodensitometric analysis of metaphyseal trabecular part of tibia from 5 week-old and 4 week-old LRP5-KO mice treated with vehicle or LiCl (200 mg/kg/d) during 15 days are given in FIGS. 4 and 5, respectively.

FIG. 4 shows that LiCL is able to significantly increase BV/TV and Tb.N. in LRP5 KO mice as compared to untreated control (vehicle n=9 and LiCl n=4)

FIG. 5 illustrates that LiCL is able to significantly increase BV/TV, Tb.N. and Tb.th. in LRP5 KO mice as compared to untreated control (vehicle n=5 and LiCl n=4)

Therefore, osteopenia LRP5 gene null-induced is partly restored by LiCl treatment

3-5—Three Dimensional Reconstruction of Bone Tissue:

3-D reconstruction of a representative trabecular metaphyseal part of tibia from 4 week-old LRP5-KO mice treated, for 15 days, with vehicle or LiCl is shown in FIG. 6.

As illustrated in FIGS. 6B (vehicle) and 6D (LiCl), magnification of trabecular part of tibia shows the increase of bone volume, an trabecular thickness after LiCl treatment.

While the invention has been described in terms of the various preferred embodiments, the skilled artisan will appreciate that various modifications, substitutions, omissions and changes may be made without departing from the scope thereof. Accordingly, it is intended that the present invention be limited by the scope of the following claims, including equivalents thereof.

### REFERENCES

Baron et al. Primer on the metabolic bone diseases and disorders of mineral metabolism, 4^{th} edition, Ed. Farus M. J., Raven Press NY.

Remington's Pharmaceutical Sciences, Mack Publication Co., Easton, Pa., latest Edition.

van de Wetering et al. (1997) Cell 88, 789-799

Kato et al. (2002) J. Cell. Biol. 157, 303-314

Doble, B. W. and Woodgett, J. R. (2003). J. Cell Sciences 116, 1175-1186

Fisher, L. et al. (2002) J. Cell Biochem. 84, 816-831

Ortega, M. A. et al. (2002) Bioorg. Med. Chem. 10, 2177-2184

Witherington, J. et al. (2003) Bioorg. Med. Chem. 13, 1581-1584

Sampath et al. (1987) PNAS 84, 7109-7113

Tencer et al. (1989) J. Biomed. Mat. Res. 23, 571-589

1. A method for preventing and/or treating a bone-related disease in a mammal in need of such treatment, wherein said method comprises:
administering to said mammal an effective amount of a pharmaceutical composition comprising at least one GSK-3β inhibitor.

2. A method for preventing and/or treating a bone-related disease in a mammal in need of such treatment, wherein said method comprises:
administering to said mammal a pharmaceutically effective amount of at least one GSK-3β inhibitor.

3. The method according to claim 1 or 2, wherein said mammal is a human.

4. The method according to claim 1 or 2, wherein said bone-related disease is selected from disorders of mineral metabolism, disorders of parathyroid hormone secretion and/or activity, metabolic bone disorders comprising osteoporosis, vitamin D related disorders, renal bone diseases, hypophosphatasia, dysplastic disorders, infiltrative disorders, extra-skeletal calcification and ossification.

5. The method according to claim 4, wherein said bone-related disease is osteoporosis.

6. The method according to claim 1 or 2, wherein said at least one GSK-3β inhibitor is selected from: lithium, bivalent zinc, beryllium, aloisines, hymenialdisine, indirubins, maleimides, muscarinic agonists, pyrazolo[3,4-b]quinoxalines, 5-aryl-pyrazolo[3,4-b]pyridazines, and functional derivatives thereof.

7. The method according to claim 5, wherein said at least one GSK-3β inhibitor is lithium.

8. A method for selecting a compound useful for preventing and/or treating a bone-related disease in a mammal in need of such treatment, wherein said method comprises:
a) testing the ability of a candidate compound to inhibit GSK-3β activity in vitro and/or in vivo; and
b) if said candidate compound inhibits GSK-3β activity, selecting said compound.

9. The method according to claim 8, further comprising purifying said compound.

10. The method according to claim 8, wherein said mammal is a human.

11. The method according to claim 8, wherein said bone-related disease is selected from disorders of mineral metabolism, disorders of parathyroid hormone secretion and/or activity, metabolic bone disorders comprising osteoporosis, vitamin D related disorders, renal bone diseases, hypophosphatasia, dysplastic disorders, infiltrative disorders, extra-skeletal calcification and ossification.

12. The method according to claim 11, wherein said bone-related disease is osteoporosis.
